(19) United States
(12) Patent Application Publication
(10) Pub. No.: US 2025/0113952 A1
(43) Pub. Date: Apr. 10, 2025

(54) BATH VANITY ASSEMBLY

(71) Applicant: AS America, Inc., Piscataway, NJ (US)

(72) Inventors: Jean-Jacques L'Henaff, New Canaan, CT (US); Ki Bok Song, Plainview, NY (US); Greg Reinecker, Brooklyn, NY (US); Jacob Nitz, Brooklyn, NY (US); Paul Flowers, London (GB)

(73) Assignee: AS America, Inc., Piscataway, NJ (US)

(21) Appl. No.: 18/695,486

(22) PCT Filed: Sep. 26, 2022
(86) PCT No.: PCT/US2022/044685

§ 371 Date: Mar. 26, 2024

Related U.S. Application Data
(60) Provisional application No. 63/248,721, filed on Sep. 27, 2021.

(51) Int. Cl. A47K 1/08; A46B 17/06; A61L 2/10

(52) U.S. Cl. CPC A47K 1/08; A46B 17/065; A61L 2/10; A61L 2202/11; A61L 2202/122; A61L 2202/20

(57) ABSTRACT

A bath vanity assembly comprising a countertop configured to receive a sink basin; and a power accessory, wherein the countertop comprises an aperture configured to receive the power accessory, the power accessory is configured to be positioned in the aperture in an inserted state and in an extended state, in the inserted state, a top surface of the power accessory is positioned adjacent to the countertop, and in the extended state, a top surface of the power accessory is positioned a distance above the countertop to provide access to a power feature positioned below the power accessory top surface.

The disclosure related to a bath vanity assembly comprising one or more power accessories.

### BACKGROUND

Bath vanities may provide for a sink basin and a faucet, and may comprise storage space comprising doors or drawers. Bathroom space is limited, therefore efficient use of a bath vanity countertop space is highly desired.

### SUMMARY

Accordingly, disclosed is a bath vanity assembly comprising a countertop configured to receive a sink basin; and a power accessory, wherein the countertop comprises an aperture configured to receive the power accessory, the power accessory is configured to be positioned in the aperture in an inserted state and in an extended state, in the inserted state, a top surface of the power accessory is positioned adjacent to the countertop, and in the extended state, a top surface of the power accessory is positioned a distance above the countertop to provide access to a power feature positioned below the power accessory top surface.

Also disclosed is a power accessory associated with a movement assembly, wherein the movement assembly is configured to move the power accessory from an inserted state to an extended state and from the extended state to the inserted state.

In some embodiments, the movement assembly is configured to move the power accessory from the inserted state to a first extended state, from the first extended state to a second extended state, from the second extended state to the first extended state, and from the first extended state to the inserted state.

In some embodiments, the movement assembly is configured to move the power accessory from the inserted state to a first extended state, from the first extended state to a second extended state, and from the second extended state directly to the inserted state.

### BRIEF DESCRIPTION OF THE DRAWINGS

This disclosure is illustrated by way of example and not by way of limitation in the accompanying FIGURES. For simplicity and clarity of illustration, features illustrated in the figures are not necessarily drawn to scale. For example, the dimensions of some features may be exaggerated relative to other features for clarity. Further, where considered appropriate, reference labels have been repeated among the FIGURES to indicate corresponding or analogous elements.

FIG. 1A and FIG. 1B provide views of a bath vanity assembly, according to some embodiments.

FIG. 1C shows a power accessory in an inserted position, according to an embodiment.

FIG. 1D shows a power accessory in a partially extended position, according to an embodiment.

FIG. 1E shows a power accessory in a fully extended position, according to an embodiment.

### DETAILED DISCLOSURE

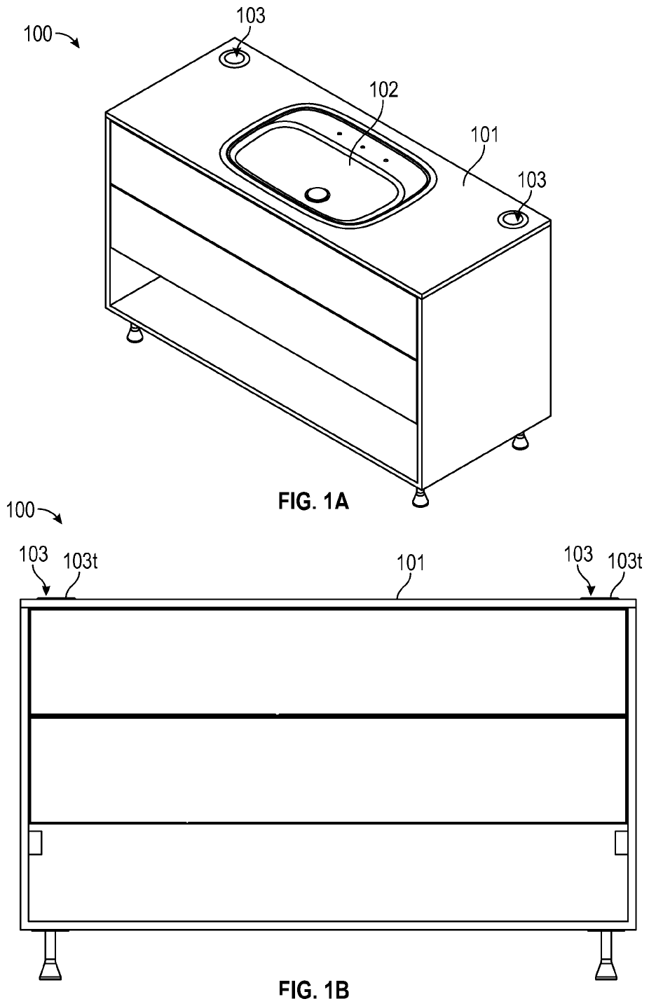

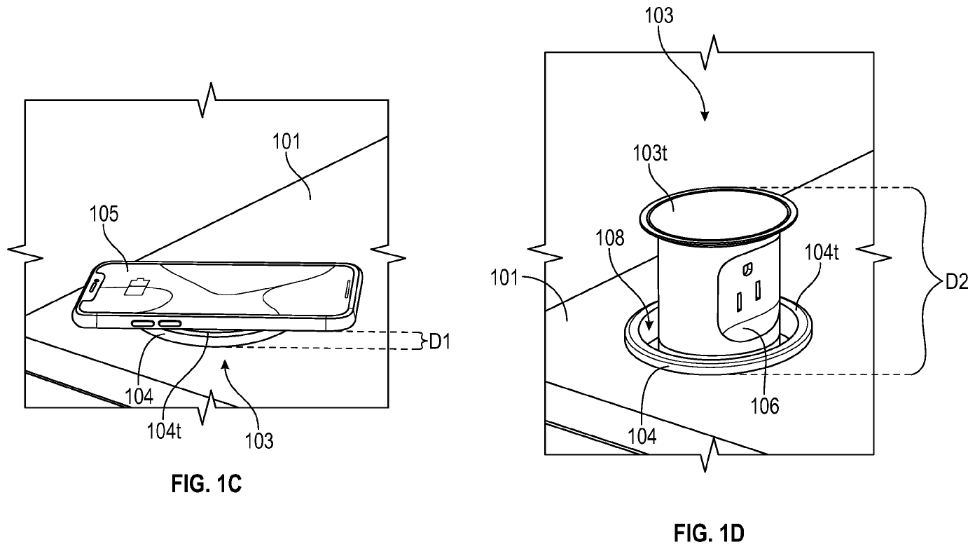

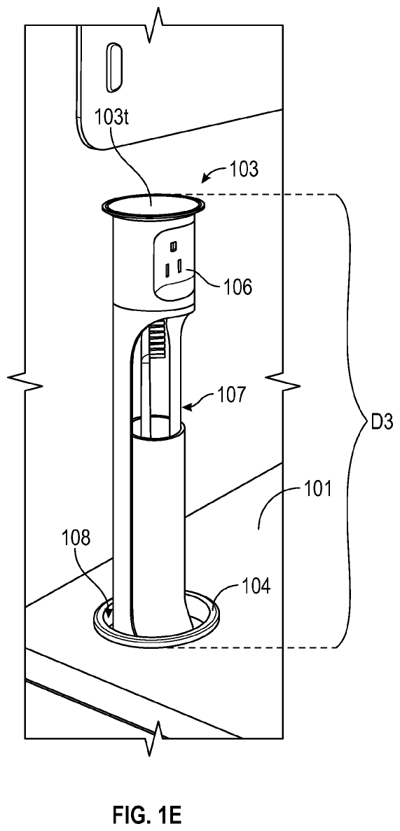

FIG. 1A and FIG. 1B provide views of bath vanity assembly 100, according to some embodiments. Assembly 100 comprises sink basin 102 positioned in countertop 101. Assembly 100 also comprises power accessories 103 positioned in countertop 101. Power accessories 103 are shown in an inserted position. As shown in front view, FIG. 1B, top surface 103t of power accessories 103 extends a distance above a top surface of countertop 101 in an inserted position.

FIG. 1C shows a view of smartphone device 105 placed on power accessory 103 in an inserted, or stored position, according to an embodiment. Countertop 101 comprises raised lip 104. In an inserted position, power accessory top surface 103t may be about flush with a top surface or edge 104t of raised lip 104. Raised lip 104 may serve to prevent any water from entering power accessory 103. Power accessory 103 is configured to inductively (wirelessly) charge smartphone device 105 when device 105 is in contact with surface 103t. A bottom surface of device 105 is positioned adjacent countertop 101, at a distance D1 above countertop 101.

FIG. 1D shows a view of power accessory 103 in a partially (first) extended position, according to an embodiment. Top surface 103t is positioned a distance D2 above countertop 101. Power accessory 103 may be configured to partially extend by pushing surface 103t a first time. Electrical outlet 106 is provided in a partially extended position.

FIG. 1E shows power accessory 103 in a fully (second) extended position, according to an embodiment. Top surface 103t is positioned a distance D3 above countertop 101. Power accessory 103 may be configured to fully extend by pushing surface 103t a second time. In a fully extended position, sanitization chamber 107 is available. Power accessory 103 is configured to move vertically within aperture 108. Raised lip 104 is positioned about aperture 108. Sanitization chamber 107 may be configured to receive a toothbrush, a retainer, dentures, and the like. Sanitization chamber may include one or more ultraviolet (UV) lights configured to sanitize an object disposed therein.

In some embodiments, a countertop may be positioned on a vanity cabinet comprising a space therein and having doors and/or drawers. A countertop may comprise one or more apertures to receive one or more power accessories. An aperture may have a raised lip positioned about it. A raised lip may be integral with a countertop, or alternatively, a raised lip may be part of a power accessory assembly. A countertop may be configured to have a sink basin positioned in or on it.

A power accessory assembly may comprise a movement assembly. In other words, a power accessory may be associated with a movement assembly. A power accessory assembly may comprise a chamber having a wall thereabout, not unlike a click-pen mechanism. A power accessory assembly may comprise a push-push mechanism, a click-pen mechanism, and the like. A power accessory assembly may be a complete part ready to be installed in an aperture of a countertop. A power accessory assembly may comprise a battery power source, or may have a wire and plug configured to plug directly into a building power supply.

In some embodiments, a movement assembly configured to assist in moving a power accessory between inserted and extended positions may include a resilient member and a detent. A resilient member may be configured to resist a person's force when one pushes a power accessory top surface to place a power accessory in an inserted position, and then can move to an extended position when the person presses the power accessory top surface again. In one or more embodiments of the disclosure, a resilient member can include a compression spring, a tension spring, or a torsion spring. Alternatively, rather than employing a spring, a resilient member can include any material that can deform and absorb energy in the presence of a force and revert back to its original form once the force is removed. A detent can include a feature configured to mechanically resist or arrest motion of an object until the detent is released. A detent can act to mechanically resist and arrest the motion of a power accessory that would otherwise be returned back to an extended position.

In other embodiments, a power accessory assembly may comprise no movement assembly, and may simply be moved from an inserted position to an extended position by pulling and pushing. In such an embodiment, an assembly may comprise an O-ring and/or a stop feature to provide for counter-force and to prevent as assembly from being pulled out or pushed in too far. Such an assembly may comprise a “chamber within a chamber” with one or more O-rings positioned between chamber walls.

In some embodiments, a raised lip is configured such that in an inserted state, a top surface of a power accessory is positioned about flush with a top surface or edge of the lip, adjacent the countertop and at a distance above the countertop. A top surface of a power accessory may be parallel with a countertop in an inserted or in an extended state. In some embodiments, in an inserted state, a distance of a power accessory top surface from a countertop surface may be from any of about 0.2 cm, about 0.4 cm, about 0.6 cm, about 0.8 cm, about 1.0 cm, about 1.2 cm, about 1.4 cm, about 1.6 cm, about 1.8 cm, about 2.0 cm, or about 2.2 cm, to any of about 2.4 cm, about 2.6 cm, about 2.8 cm, about 3.0 cm, about 3.2 cm, about 3.4 cm, or more.

This distance may be a distance D1 as shown in FIG. 1C. Distance D2 of FIG. 1D is greater than distance D1, for example about the width of a single or a double outlet. Distance D3 of FIG. 1E will be greater than distance D2, for example about the length of a toothbrush.

In some embodiments, pushing a power accessory top surface a first time will cause it to move to an extended state, and pushing it a second time will cause it to return to an inserted state. In some embodiments, pushing a power accessory top surface a first time will cause it to move to a first extended state, pushing it a second time will cause it to move to a second extended state, pushing it a third time will cause it to move to the first extended state, and pushing it a fourth time will cause it to move back to the inserted state.

In some embodiment, pushing a power accessory top surface a first time will cause it to move to a first extended state, pushing it a second time will cause it to move to a second extended state, and pushing it a third time will cause it to move directly back to the inserted state.

In some embodiments, a top surface of a power accessory may itself comprise a power feature configured to inductively (wirelessly) charge a device, for example a smartphone, tablet, and the like. In some embodiments, a power accessory may comprise a power feature positioned below a top surface, which may be available when the power accessory is in an extended state. In some embodiments, one or more electrical outlets may be available in a first extended state, and one or more sanitization chambers may be available in a second extended state.

In some embodiments, a sanitization chamber may comprise an ultraviolet (UV) light configured to disinfect a toothbrush, retainer, denture, etc. In some embodiments, a UV light may be disabled when a power accessory is in an extended state. In some embodiments, a UV light may be configured to emit UV when a power accessory is placed in an inserted state. A UV light may be configured to emit for a prescribed period of time to kill pathogens. For example, a time period to emit UV may be about 1 minute, about 2 minutes, about 3 minutes, or more. In some embodiments, a time period to emit UV may be less than about 1 minute.

In some embodiments, a power accessory may be associated with a timer or clock, and a UV light may be configured to emit UV periodically, for example at least once per day, twice per day, three times per day, once per week, twice per week, etc., in order to prevent any bacterial growth. In some embodiments, a sterilization chamber may comprise one or more small holes positioned in a bottom thereof, configured to allow for water drainage and for air flow. In some embodiments, a power accessory may comprise multiple sanitization chambers. In some embodiments, a single sanitization chamber may be configured to receive multiple items, e.g. multiple toothbrushes. In some embodiments, a chamber may be configured to store an item like a toothbrush, but contain no sanitization feature (like UV).

The terms “coupled” or “connected” may mean that an element is “attached to” or “associated with” another element. Coupled or connected may mean directly coupled or coupled through one or more other elements. An element may be coupled to an element through two or more other elements in a sequential manner or a non-sequential manner. The term “via” in reference to “via an element” may mean “through” or “by” an element. Coupled or connected or “associated with” may also mean elements not directly or indirectly attached, but that they “go together” in that one may function together with the other.

The term “towards” in reference to a of point of attachment, may mean at exactly that location or point or, alternatively, may mean closer to that point than to another distinct point, for example “towards a center” means closer to a center than to an edge.

The term “like” means similar and not necessarily exactly like. For instance “ring-like” means generally shaped like a ring, but not necessarily perfectly circular.

The articles “a” and “an” herein refer to one or to more than one (e.g. at least one) of the grammatical object. Any ranges cited herein are inclusive. The term “about” used throughout is used to describe and account for small fluctuations. For instance, “about” may mean the numeric value may be modified by ±0.05%, ±0.1%, ±0.2%, ±0.3%, ±0.4%, ±0.5%, ±1%, ±2%, ±3%, ±4%, ±5%, ±6%, ±7%, ±8%, ±9%, ±10% or more. All numeric values are modified by the term “about” whether or not explicitly indicated. Numeric values modified by the term “about” include the specific identified value. For example “about 5.0” includes 5.0.

The term “substantially” is similar to “about” in that the defined term may vary from for example by ±0.05%, ±0.1%, ±0.2%, ±0.3%, ±0.4%, ±0.5%, ±1%, ±2%, ±3%, ±4%, ±5%, ±6%, ±7%, ±8%, ±9%, ±10% or more of the definition; for example the term “substantially perpendicular” may mean the 90° perpendicular angle may mean “about 90°”. The term “generally” may be equivalent to “substantially”.

Features described in connection with one embodiment of the disclosure may be used in conjunction with other embodiments, even if not explicitly stated.

Embodiments of the disclosure include any and all parts and/or portions of the embodiments, claims, description and FIGURES. Embodiments of the disclosure also include any and all combinations and/or sub-combinations of embodiments.

1. A bath vanity assembly comprising
a countertop configured to receive a sink basin; and
a power accessory,
wherein
the countertop comprises an aperture configured to receive the power accessory,
the power accessory is configured to be positioned in the aperture in an inserted state and in an extended state,
in the inserted state, a top surface of the power accessory is positioned adjacent to the countertop, and
in the extended state, a top surface of the power accessory is positioned a distance above the countertop to provide access to a power feature positioned below the power accessory top surface.

2. The bath vanity assembly according to claim 1, wherein a top surface of the power accessory comprises a power feature configured to provide for wireless charging of a device.

3. The bath vanity assembly according to claim 1, wherein the power feature positioned below the power accessory top surface comprises an electrical outlet.

4. The bath vanity assembly according to claim 1, wherein the power feature positioned below the power accessory top surface comprises a sanitization chamber, the sanitization chamber comprising a UV light source.

5. The bath vanity assembly according to claim 4, wherein the sanitization chamber is configured to receive a toothbrush.

6. The bath vanity assembly according to claim 4, wherein the UV light source is configured to be disabled if the power accessory is in the extended state.

7. The bath vanity assembly according to claim 1, wherein the power accessory is configured to be positioned in a first extended state to provide access to a first power feature.

8. The bath vanity assembly according to claim 7, wherein the power accessory is configured to be positioned in a second extended state to provide access to a second power feature.

9. The bath vanity assembly according to claim 1, wherein the countertop comprises a raised lip positioned about the aperture.

10. The bath vanity assembly according to claim 9, wherein in the inserted state, the power accessory top surface is about flush with a top surface or edge of the raised lip.

11. The bath vanity assembly according to claim 1, wherein the power accessory is configured to be moved from the inserted state to the extended state by pushing the power accessory top surface.

12. The bath vanity assembly according to claim 1, wherein the power accessory is configured to be moved from the inserted state to a first extended state by pushing the power accessory top surface a first time, and to be moved from the first extended state to a second extended state by pushing the power accessory top surface a second time.

13. The bath vanity assembly according to claim 1, wherein the power accessory is configured to be moved from the extended state to the inserted state by pushing the power accessory a second time.

14. The bath vanity assembly according to claim 1, wherein the power accessory is associated with a movement assembly configured to move the power accessory from the inserted state to the extended state and from the extended state to the inserted state.

15. The bath vanity assembly according to claim 14, wherein the movement assembly is configured to move the power accessory from the inserted state to a first extended state, from the first extended state to a second extended state, from the second extended state to the first extended state, and from the first extended state to the inserted state.

16. The bath vanity assembly according to claim 14, wherein the aperture comprises a movement assembly configured to move the power accessory from the inserted state to a first extended state, from the first extended state to a second extended state, and from the second extended state to the inserted state.

17. The bath vanity assembly according to claim 14, wherein the movement assembly comprises a push-push assembly or a click-pen assembly.

18. The power accessory associated with a movement assembly according to claim 14.

19. The power accessory according to claim 18, wherein the power accessory top surface comprises a wireless charger.

20. The power accessory according to claim 19, wherein the power accessory comprises an electrical outlet and/or a sanitization chamber positioned below the power accessory top surface.
